(19) United States
(12) Patent Application Publication
(10) Pub. No.: US 2012/0009244 A1
(43) Pub. Date: Jan. 12, 2012

(54) NEUROD1 GENE EXPRESSION IN NON-ENDOCRINE PANCREATIC EPITHELIAL CELLS (NEPECs)

(75) Inventors: Masayuki Shimoda, Irving, TX (US); Shuyuan Chen, Allen, TX (US); Hirofumi Noguchi, Kitu-ku (JP); Shinichi Matsumoto, Arlington, TX (US); Paul A. Grayburn, Dallas, TX (US)

(73) Assignee: BAYLOR RESEARCH INSTITUTE, Dallas, TX (US)

(21) Appl. No.: 13/071,458

(22) Filed: Mar. 24, 2011

Related U.S. Application Data
(60) Provisional application No. 61/317,159, filed on Mar. 24, 2010.

(51) Int. Cl. A61K 9/127; C12N 5/10; A61P 3/08; A61K 31/7105; A61P 3/10; A61P 5/50; C12N 15/85; C12Q 1/02

(52) U.S. Cl. 424/450; 435/320.1; 435/371; 435/29; 514/44 R

(57) ABSTRACT

The introduction of the human NeuroD1 gene into human non-endocrine pancreatic epithelial cells (NEPECs) for producing insulin producing cells in vitro is described herein. Cytokeratin19 (CK19) positive NEPECs were transfected with plasmids encoding human NeuroD1 gene under human CK19 promoter. On characterization following the induction it was found that NEPEC+ND strongly expressed NeuroD1 and insulin mRNA. The ratio of NeuroD1 and human insulin positive cells in NEPEC+ND was significantly higher than NEPEC. Human insulin and C-peptide levels in culture media in NEPEC+ND were significantly higher than NEPEC. The findings demonstrate that human NeuroD1 under control of the CK19 promoter induces the differentiation of CK19 positive NEPECs into insulin producing cells.

### CROSS-REFERENCE TO RELATED APPLICATIONS

This patent application is a non-provisional application of U.S. provisional patent application 61/317,159 filed on Mar. 24, 2010 and entitled “NeuroD1 Gene Expression in Non-Endocrine Pancreatic Epithelial Cells (NEPECs)” which is hereby incorporated by reference in its entirety.

### STATEMENT OF FEDERALLY FUNDED RESEARCH

This invention was made with U.S. Government support under Contract Nos. R01 HL072430-01 and 2P01 DK58398 awarded by the National Institutes of Health (NIH). The government has certain rights in this invention

### TECHNICAL FIELD OF THE INVENTION

The present invention relates in general to the field of gene delivery, and more particularly, to the introduction of NeuroD1 gene to direct differentiation of cytokeratin 19-positive human pancreatic non-endocrine cells (NEPECs) into insulin producing cells.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with gene delivery methods to promote in vivo production of insulin.

U.S. Pat. No. 7,323,165 (German, 2008) relates to the production of islet cells and insulin in a subject by providing for expression of an islet transcription factors in the pancreas of the subject, by for example, introduction of nucleic acid encoding the transcription factor neurogenin3 or a factor that induces neurogenin3 expression. The present invention also relates to methods for using an islet transcription factor gene and the islet transcription factor polypeptide to alter cellular differentiation in culture or in vivo to produce new β-cells to treat patients with diabetes mellitus.

U.S. Pat. No. 7,374,390 issued to Oh, et al. (2008), discloses compositions and methods of use to normalize blood glucose levels of patients with type 2 diabetes. The invention includes a plasmid comprising a chicken 0 actin promoter and enhancer; a modified GLP-1 (7-37) cDNA (pβGLP1), carrying a furin cleavage site, which is constructed and delivered into a cell for the expression of active GLP-1.

### SUMMARY OF THE INVENTION

The present invention describes the introduction the human NeuroD1 gene into human non-endocrine pancreatic epithelial cells (NEPECs) to promote insulin producing cells.

In one embodiment the present invention provides a method of treating diabetes in a mammal in need thereof comprising administering a therapeutically effective amount of an isolated nucleic acid including a sequence encoding a NeuroD1 gene to one or more non-endocrine pancreatic epithelial cells of the mammal and expressing a NeuroD1 protein in the cell, wherein the NeuroD1 protein comprises the amino acid sequence set forth in SEQ ID NOS: 10 or 12, thereby treating the diabetes of the mammal. In one aspect of the method described herein the non-endocrine pancreatic epithelial cells are cytokeratin 19+ positive cells. In another aspect the non-endocrine pancreatic epithelial cells are human cytokeratin 19+ positive cells and the NeuroD1 is a human NeuroD1.

In yet another aspect of the method of the present invention the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a perfluorocarbon gas and the pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells. In related aspects the inducible promoter comprises a CK19 promoter, more specifically a human CK19 promoter and the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

Another embodiment of the instant invention discloses a gene construct or a plasmid DNA comprising an isolated nucleic acid including a sequence encoding a NeuroD1 gene, wherein the NeuroD1 gene expresses a NeuroD1 protein comprising the amino acid sequence set forth in SEQ ID NOS: 10 or 12 in one or more cells and a constitutive promoter sequence or an inducible promoter sequence operably linked to the NeuroD1 gene. In specific aspects of the composition disclosed herein the NeuroD1 is a human NeuroD1, the inducible promoter is a human CK19 promoter and the one or more cells are human cytokeratin 19+ non-endocrine pancreatic epithelial cells.

Further the present invention also describes a composition for islet transplantation comprising one or more human cytokeratin 19+ non-endocrine pancreatic epithelial cells, wherein the cells are transfected with a NeuroD1 gene under the control of a constitutive promoter sequence or an inducible promoter sequence operably linked to the NeuroD1 gene. In one aspect the NeuroD1 gene expresses a NeuroD1 protein comprising the amino acid sequence set forth in SEQ ID NOS: 10 or 12 in the one or more cells. In another aspect the composition is used for treating diabetes, for promoting euglycemia or for making one or more glucose responsive cells.

In yet another embodiment the present invention provides a composition for making sugar responsive cells comprising a microbubble capable of delivering to non-endocrine pancreatic epithelial cells one or more isolated nucleic acids comprising a plasmid DNA encoding a NeuroD1 gene under the control of a constitutive promoter sequence or an inducible promoter sequence and expressing a NeuroD1 protein in the cells, wherein the NeuroD1 protein comprises the amino acid sequence set forth in SEQ ID NOS: 10 or 12, wherein the microbubbles comprise lipids that release the plasmid by ultrasound disruption into the non-endocrine pancreatic epithelial cells. In one aspect the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a perfluorocarbon gas and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells. In one aspect the inducible promoter is a CK19 promoter. In another aspect the CK19 promoter is a human CK19 promoter. In yet another aspect the efficacy of NeuroD1 expression is determined by increased responsiveness to blood sugar as measured by insulin release by the non-endocrine pancreatic epithelial cells. In a specific aspect the NeuroD1 is a human NeuroD1.

In one embodiment the present invention describes a method of treating diabetes or promoting euglycemia in a patient comprising the steps of: identifying the patient in need of treatment against the diabetes or promotion of the euglycemia, and injecting an effective amount of a microbubble capable of delivering to non-endocrine pancreatic epithelial cells one or more isolated nucleic acids comprising a plasmid DNA encoding a NeuroD1 gene, wherein the microbubbles comprise lipids that release the plasmid by ultrasound disruption into the non-endocrine pancreatic epithelial cells. In one aspect the non-endocrine pancreatic epithelial cells are cytokeratin 19+ positive cells, more specifically human cytokeratin 19+ positive cells. In another aspect the NeuroD1 is a human NeuroD1.

In yet another aspect the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a perfluorocarbon gas and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells.

In one aspect the inducible promoter comprises a CK19 promoter, wherein the CK19 promoter is a human CK19 promoter. In another aspect the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

In another embodiment the present invention is a method of providing an insulin-producing cell, the method comprising: providing one or more isolated non-endocrine pancreatic epithelial cell, transfecting the cells with an isolated nucleic acid encoding a NeuroD1 polypeptide comprising a sequence that is at least 95% identical to SEQ ID NOS: 10 or 12, wherein the polypeptide can increase, cause transcription or both of the insulin gene in the non-endocrine pancreatic epithelial cell, and assaying insulin production in the cells, thereby providing an insulin-producing cell. In one aspect of the method the non-endocrine pancreatic epithelial cells are cytokeratin 19+ positive cells, more specifically human cytokeratin 19+ positive cells. In another aspect the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a gas, more specifically a perfluorocarbon gas, and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells. In yet another aspect the inducible promoter comprises the CK19 promoter selected from a human CK19 promoter.

In one aspect of the method of the present invention the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid. In another aspect the NeuroD1 is a human NeuroD1. Yet another aspect describes an in vivo insulin producing cell generated by the method of the present invention.

Yet another embodiment of the instant invention provides for a method of treating one or more non-endocrine pancreatic epithelial cells, islets, or both transplanted in a liver with an ultrasound-targeted microbubble destruction (UTMD) technique, wherein the treatments results in increased insulin production, increased glucose responsiveness or both comprising the step of delivering via ultrasound-targeted microbubble destruction (UTMD) a vector comprising one or more pre-assembled liposome naked plasmid DNA (pDNA) microbubble complexes, wherein the microbubble comprises a lipid shell enclosing a gas and the pDNA comprising a constitutive promoter sequence or an inducible promoter sequence operably linked to a NeuroD1 gene, wherein an ultrasound disruption of the one or more microbubbles in the pancreas delivers the NeuroD1 gene into the transplanted non-endocrine pancreatic epithelial cells, islets or both resulting in an expression of a NeuroD1 protein in the non-endocrine pancreatic epithelial cells, islets or both, wherein the NeuroD1 protein comprises the amino acid sequence set forth in SEQ ID NOS: 10 or 12. The method as described herein further comprises the step of determining increased responsiveness to blood sugar by measuring insulin release by the transplanted non-endocrine pancreatic epithelial cells, the islets or both.

In one aspect the efficacy of the transplantation of the one or more non-endocrine pancreatic epithelial cells, islets, or both is measured by improved revascularization, improved islet cell function, increased vessel density or combinations thereof. In specific aspects the NeuroD1 is a human NeuroD1, the inducible promoter is a human CK19 promoter, the gas is a perfluorocarbon gas and the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid. Finally in one aspect a non-endocrine pancreatic epithelial cell, an islet or both with increased glucose responsiveness, increased insulin production or both made by the method of the present invention is disclosed.

### BRIEF DESCRIPTION OF THE DRAWINGS

For a more complete understanding of the features and advantages of the present invention, reference is now made to the detailed description of the invention along with the accompanying figures and in which:

FIG. 1 shows the gene expression analysis of NEPC, NEPEC and NEPEC+ND by RT-PCR. All cells were harvested at day 7 after starting of induction of hND. Human islets were used as a control;

FIG. 2A-2F show representative micrographs of NEPEC+ND (FIG. 2A-2C) or NEPEC (FIG. 2D-2F). The cells were labeled with anti-NeuroD1 antibody (Red) or anti-insulin antibody (Green). All cell nuclei were stained with DAPI (Blue). Original magnifications: X100 (FIGS. 2A and 2C), X200 (FIGS. 2B and 2D). The ratio of human NeuroD1 (FIG. 2E) or Insulin (FIG. 2F) positive cells in NEPEC (white bar) and NEPEC+ND (black bar). Data are mean values±SE. Asterisks: p<0.01; and

FIGS. 3A and 3B show Human insulin (FIG. 3A) and C-peptide (FIG. 3B) levels of NEPC (white bar), NEPEC (stripe bar) and NEPEC+ND (black bar) in culture media. The samples were collected after 24 hours culture at day 7 after starting the induction of human NeuroD1. Data are mean values±SE. Asterisks: p<0.01.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

The term “diabetes” as described in embodiments of the present invention refers to the chronic disease characterized by relative or absolute deficiency of insulin that results in glucose intolerance. The term “diabetes” is also intended to include those individuals with hyperglycemia, including chronic hyperglycemia, hyperinsulinemia, impaired glucose homeostasis or tolerance, and insulin resistance.

The term “insulin” as used herein shall be interpreted to encompass insulin analogs, natural extracted human insulin, recombinantly produced human insulin, insulin extracted from bovine and/or porcine sources, recombinantly produced porcine and bovine insulin and mixtures of any of these insulin products. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution.

The term “islet cell (s)” as used throughout the specification is a general term to describe the clumps of cells within the pancreas known as islets, e.g., islets of Langerhans. Islets of Langerhans contain several cell types that include, e.g., β-cells (which make insulin), α-cells (which produce glucagons), γ-cells (which make somatostatin), F cells (which produce pancreatic polypeptide), enterochromaffin cells (which produce serotonin), PP cells and D1 cells. The term “stem cell” is an art recognized term that refers to cells having the ability to divide for indefinite periods in culture and to give rise to specialized cells. Included within this term are, for example, totipotent, pluripotent, multipotent, and unipotent stem cells, e.g., neuronal, liver, muscle, and hematopoietic stem cells.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “vector” is used in reference to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “promoter” is defined as a DNA sequence recognized by the synthetic machinery of the cell, or introduced synthetic machinery, required to initiate the specific transcription of a gene. As used herein, the term “under transcriptional control” or “operatively linked” is defined as the promoter is in the correct location and orientation in relation to the nucleic acid to control RNA polymerase initiation and expression of the hVEGF gene.

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “transfection” as used herein refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “in vivo” refers to being inside the body. The term “in vitro” used as used in the present application is to be understood as indicating an operation carried out in a non-living system.

The term “liposome” as used herein refers to a capsule wherein the wall or membrane thereof is formed of lipids, especially phospholipid, with the optional addition therewith of a sterol, especially cholesterol.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes the introduction of the human NeuroD1 gene into human non-endocrine pancreatic epithelial cells (NEPECs) and promotion of insulin producing cells in vitro. Cytokeratin19 (CK19) positive NEPECs were transfected with plasmids encoding human NeuroD1 gene under human CK19 promoter. On characterization following the induction it was found that NEPEC+ND strongly expressed NeuroD1 and insulin mRNA. The ratio of NeuroD1 and human insulin positive cells in NEPEC+ND was significantly higher than NEPEC. Human insulin and C-peptide levels in culture media in NEPEC+ND were significantly higher than NEPEC. The findings demonstrate that human NeuroD1 under control of the CK19 promoter induces the differentiation of CK19 positive NEPECs into insulin producing cells.

The present invention demonstrates that human NeuroD1 under control of the CK19 promoter can induce the differentiation of CK19 positive non-endocrine pancreatic epithelial cells (NEPECs) into insulin producing cells. It has been reported that the human pancreatic non-endocrine fraction, which remains after islet isolation, can be differentiated toward beta cells. However, the optimal method to accomplish this has not been established. The present invention addresses this issue by introducing the human NeuroD1 gene into human NEPECs and promotes insulin producing cells in vivo or in vitro.

One embodiment of the present invention discloses a method of administering an isolated nucleic acid including a sequence encoding a NeuroD1 gene to one or more non-endocrine pancreatic epithelial cells of the mammal and expressing a NeuroD1 protein in the cell. The isolated nucleic acid is delivered via ultrasound-targeted microbubble destruction (UTMD) using a vector comprising one or more pre-assembled liposome plasmid DNA (pDNA) microbubble complexes. The microbubble comprises a lipid shell enclosing a gas and a pDNA comprising a constitutive promoter sequence or an inducible promoter sequence operably linked to the NeuroD1 gene. An ultrasound disruption of the one or more microbubbles in the pancreas delivers the NeuroD1 gene into the non-endocrine pancreatic epithelial cells. UTMD methods and compositions for gene delivery have been previously described by the inventors in WIPO patent application No. PCT/US09/64467 and U.S. patent application Ser. No. 61/298,824, respectively the contents of which are incorporated herein by reference.

The human pancreatic non-islet fractions were obtained from brain-dead donors and cultured in suspension for 2-3 days followed by culture with G418 for 4 days. These cells (NEPECs) were then plated on dishes. NEPECs spread into a cell monolayer within 7 days and all these cells were cytokeratin19 (CK19) positive. Seven days after plating, plasmids encoding human NeuroD1 gene under human CK19 promoter were transfected 3 times every other day (termed NEPEC+ND). Seven days after starting induction, these cells were characterized. Seven days after starting the induction of human NeuroD1, NEPEC+ND strongly expressed NeuroD1 and insulin mRNA. The ratio of NeuroD1 positive cells in NEPEC+ND was significantly higher than NEPEC. And human insulin positive cells in NEPEC+ND were also significantly greater than NEPEC. Human insulin and C-peptide levels in culture media in NEPEC+ND were significantly higher than NEPEC.

Although islet transplantation is a promising treatment for type 1 diabetes and the success rate has increased (1, 2), donor shortage remains a major limitation. Therefore, promoting the generation of new beta cells is a potential therapy. Earlier studies suggested that beta cells can regenerate from putative stem or progenitor cells, including ductal cells (3-9). It was recently reported that the human pancreatic non-endocrine fraction, which is the remainder after islet isolation and consists of epithelial and mesenchymal cells, can differentiate into beta cells under the influence of inductive factors existing in the human fetal pancreas (10). However, the mechanism and the nature of those factors have not been elucidated. In the present invention, the inventors introduced human NeuroD1, a transcriptional factor which plays an important role during beta cell generation, into human non-endocrine pancreatic epithelial cells (NEPECs) for promoting insulin producing cells in vitro.

Plasmid constructs: The inventors constructed a plasmid expressing human NeuroD1 (hND) gene under human cytokeratin 19 (CK19) promoter lesion (pCK19-hND) as follows: A full-length cDNA of the hND was PCR amplified. The DNA was digested and then inserted into the corresponding sites of pCI Mammalian Expression Vector (Promega, WI). Then a specific cis-regulatory element (−732 to ATG) upstream of human CK19 transcription start site was amplified by PCR and replaced CMV promoter of the pCI vector (11). Cloning, isolation, and purification of this plasmid were performed by standard procedures, and sequenced to confirm that no artifactual mutations were present.

NEPECs and human NeuroD1 induction: Donor pancreata were procured from deceased multiorgan donors after obtaining consent for research through local Organ Procurement Organizations (Southwest Transplant Alliance, Dallas, Tex., LifeGift, Fort Worth, Tex.) (12). Islet isolation was performed using the semiautomated method described by Ricordi et al. with some modifications described by the inventors previously (13-17).

The pancreatic non-islet fraction was obtained from less purified islet fraction and COBE bag fraction and cultured in suspension in RPMI with 10% FBS for 2-3 days. Then, to deplete fibroblasts and residual islets, they were cultured with 40 μg/ml G418 for 4 days. Then these cells (NEPECs) were plated on Matrigel™ (BD Biosciences, CA) coated dishes. NEPECs spread into a cell monolayer on the matrix within 7 days and all these cells were CK19 positive. Seven days after plating, the pCK19-hND plasmid was transfected with lipofectamine2000 (Invitrogen, CA) every other day, totally 3 times (termed NEPEC+ND). The cells without the treatment of G418, which predominantly contained fibroblasts, were used as a control (termed non-endocrine pancreatic cells, NEPCs). They were characterized by immunohistochemistry and RT-PCR for gene expression and ELISA for human insulin and C-peptide secretion in culture media (ALPCO, NH).

The used primers for RT-PCR were as follows: human beta actin sense-CTC CAT CCT GGC CTC GCT GT (SEQ ID NO: 1), antisense-GCT GTC ACC TTC ACC GTT CC (SEQ ID NO: 2), human CK19 sense-CGA GCA GAA CCG GAA GGA TG (SEQ ID NO: 3), antisense-AGC CGC TGG TAC TCC TGA TTC (SEQ ID NO: 4), human NeuroD1 sense-GCG CTC AGG CAA AAG CCC (SEQ ID NO: 5), antisense-GCC ATT GAT GCT GAG CGG CG (SEQ ID NO: 6), human Insulin sense-CAG CCG CAG CCT TTG TGA AC (SEQ ID NO: 7), antisense-AAT GCC ACG CTT CTG CAG GG (SEQ ID NO: 8). The following antibodies were used for immunohistochemistry: rabbit anti-NeuroD1 (Millipore, MA), Guinea pig anti-insulin (Abcam, MA). The corresponding secondary antibodies conjugated with either FITC or Rhodamine (Invitrogen, CA) were used.

Statistical analysis: Data were expressed as mean±standard error. Statistically significant differences among the three groups were determined by ANOVA followed by Student's t-test with Bonferroni correction.

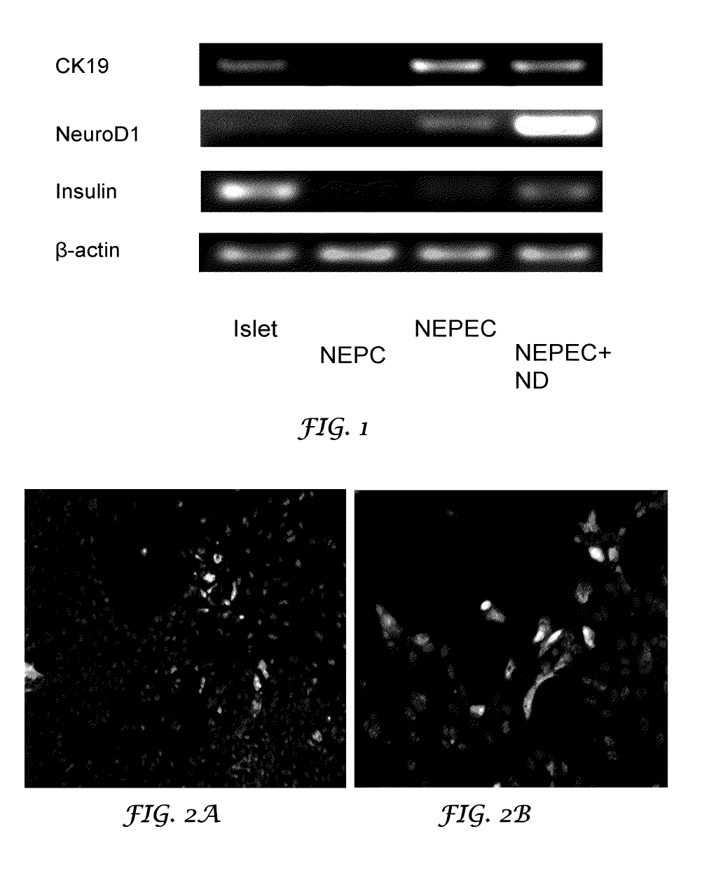

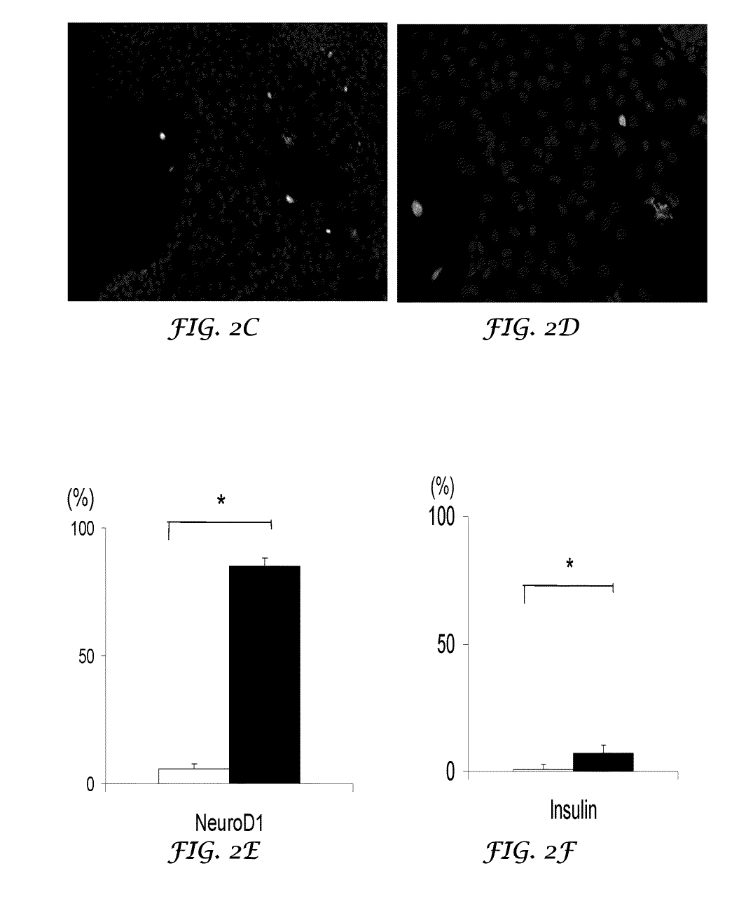

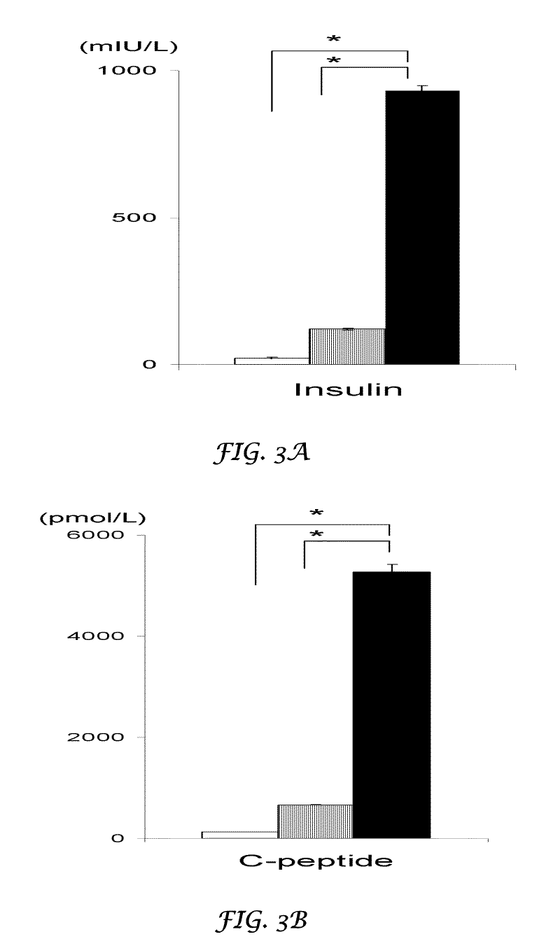

Seven days after initial induction of human NeuroD1, NEPEC+ND strongly expressed NeuroD1 and insulin mRNA (FIG. 1). At the time, most cells in NEPEC+ND expressed hND (FIGS. 2A and 2B) whereas NEPECs had a few positive cells (FIGS. 2C and 2D). The ratio of NeuroD1 positive cells in NEPEC+ND was significantly higher than NEPEC (FIG. 2E, NEPEC+ND: 85.0±3.1%; NEPEC: 5.8±2.2%, respectively). The number of human insulin positive cells in NEPEC+ND was also significantly greater than NEPEC (FIG. 2F, NEPEC+ND: 7.3±1.1%; NEPEC: 0.8±0.2%, respectively). Human insulin levels in culture media in NEPEC+ND at the same day were significantly higher than NEPEC (FIG. 3A, NEPEC+ND: 932.4±17.0 μIU/ml; NEPEC: 120.6±2.20 μIU/ml, respectively). In addition, the C-peptide level in NEPEC+ND group at the time was significantly higher than NEPEC (FIG. 3B, NEPEC+ND: 5270.5±150.9 pmol/l; NEPEC: 662.8±9.6 pmol/l, respectively). The control cells (NEPCs) were occupied by fibroblasts, so they showed very low expressions of hND, insulin and C-peptide.

The inventors have developed a CK19 promoter-NeuroD1 gene plasmid (pCK19-hND) and the transfection protocol of the present invention effectively induced NeuroD1 gene into NEPECs, which resulted in the significant increase of insulin producing cells in vitro. Although the mechanism is not known, the increase of insulin producing cells could be caused by the differentiation of NEPECs rather than the proliferation of residual beta cells because the pCK19-hND plasmid is thought to act only in CK19 positive cells. It has been shown that NEPECs can be induced to differentiate into insulin expressing cells under the influence of inductive factors present in the human fetal pancreas (10). However, the nature of those factors is still unknown. The data obtained in studies from the present invention strongly suggest that NeuroD1 is a key factor to promote differentiation. NeuroD1 is one of the class B bHLH factors and known to regulate insulin gene transcription. It is also important for the terminal differentiation of both insulin and glucagon producing islet cells (18, 19). Recent studies suggested that NeuroD1 could function to differentiate pancreatic stem/progenitor cells or even differentiating other organ's cells into beta cells (8, 20).

The contribution of the residual beta cells for insulin production is considered minimal under the conditions described herein. The present inventors used G418 to eliminate them, and beta cells are thought to change the character and lose the insulin producing function during the monolayer culture (21). In addition, human beta cells have very low proliferation capacity (22). In the protocol of the present inventors, all mesenchymal cells were also depleted before monolayer culture; strongly indicating that epithelial cells in the adult human pancreas have the differentiation potential toward endocrine cells. However, the origin of NEPECs remains unclear.

The present invention establishes a new method using human NeuroD1 gene induction under control of the CK19 promoter to induce the differentiation of CK19 positive NEPECs into insulin producing cells.

Human NeuroD1 nucleic acid sequence (NM_002500)
(SEQ ID NO: 9)
gagaacgggg agcgcacagc ctggacgcgt gcgcaggcgt caggcgcata gacctgctag cccctcagct agcggccccg
cccgcgctta gcatcactaa ctgggctata taacctgagc gcccgcgcgg ccacgacacg aggaattcgc ccacgcagga
ggcgcggcgt ccggaggccc cagggttatg agactatcac tgctcaggac ctactaacaa caaaggaaat cgaaacatga
ccaaatcgta cagcgagagt gggctgatgg gcgagcctca gccccaaggt cctccaagct ggacagacga gtgtctcagt
tctcaggacg aggagcacga ggcagacaag aaggaggacg acctcgaagc catgaacgca gaggaggact cactgaggaa
cgggggagag gaggaggacg aagatgagga cctggaagag gaggaagaag aggaagagga ggatgacgat caaaagccca
agagacgcgg ccccaaaaag aagaagatga ctaaggctcg cctggagcgt tttaaattga gacgcatgaa ggctaacgcc
cgggagcgga accgcatgca cggactgaac gcggcgctag acaacctgcg caaggtggtg ccttgctatt ctaagacgca
gaagctgtcc aaaatcgaga ctctgcgctt ggccaagaac tacatctggg ctctgtcgga gatcctgcgc tcaggcaaaa
gcccagacct ggtctccttc gttcagacgc tttgcaaggg cttatcccaa cccaccacca acctggttgc gggctgcctg
caactcaatc ctcggacttt tctgcctgag cagaaccagg acatgccccc ccacctgccg acggccagcg cttccttccc
tgtacacccc tactcctacc agtcgcctgg gctgcccagt ccgccttacg gtaccatgga cagctcccat gtcttccacg
ttaagcctcc gccgcacgcc tacagcgcag cgctggagcc cttctttgaa agccctctga ctgattgcac cagcccttcc
tttgatggac ccctcagccc gccgctcagc atcaatggca acttctcttt caaacacgaa ccgtccgccg agtttgagaa
aaattatgcc tttaccatgc actatcctgc agcgacactg gcaggggccc aaagccacgg atcaatcttc tcaggcaccg
ctgcccctcg ctgcgagatc cccatagaca atattatgtc cttcgatagc cattcacatc atgagcgagt catgagtgcc
cagctcaatg ccatatttca tgattagagg cacgccagtt tcaccatttc cgggaaacga acccactgtg cttacagtga ctgtcgtgtt
tacaaaaggc agccctttgg gtactactgc tgcaaagtgc aaatactcca agcttcaagt gatatatgta tttattgtca ttactgcctt
tggaagaaac aggggatcaa agttcctgtt caccttatgt attattttct atagctcttc tatttaaaaa ataaaaaaat acagtaaagt
ttaaaaaata caccacgaat ttggtgtggc tgtattcaga tcgtattaat tatctgatcg ggataacaaa atcacaagca ataattagga
tctatgcaat ttttaaacta gtaatgggcc aattaaaata tatataaata tatatttttc aaccagcatt ttactacttg ttacctttcc
catgctgaat tattttgttg tgattttgta cagaattttt aatgactttt tataatgtgg atttcctatt ttaaaaccat gcagcttcat
caatttttat acatatcaga aaagtagaat tatatctaat ttatacaaaa taatttaact aatttaaacc agcagaaaag tgcttagaaa
gttattgtgt tgccttagca cttctttcct ctccaattgt aaaaaaaaaa aaaaaaaaaa aaaaaaaaaa aaaattgcac aatttgagca
attcatttca ctttaaagtc tttccgtctc cctaaaataa aaaccagaat cataattttc aagagaagaa aaaattaaga gatacattcc
ctatcaaaac atatcaattc aacacattac ttgcacaagc ttgtatatac atattataaa taaatgccaa catacccttc tttaaatcaa
aagctgcttg actatcacat acaatttgca ctgttacttt ttagtctttt actcctttgc attccatgat tttacagaga atctgaagct
attgatgttt ccagaaaata taaatgcatg attttataca tagtcacaaa aatggtggtt tgtcatatat tcatgtaata aatctgagcc
taaatctaat caggttgtta atgttgggat ttatatctat agtagtcaat tagtacagta gcttaaataa attcaaacca tttaattcat
aattagaaca atagctattg catgtaaaat gcagtccaga ataagtgctg tttgagatgt gatgctggta ccactggaat cgatctgtac
tgtaattttg tttgtaatcc tgtatattat ggtgtaatgc acaatttaga aaacattcat ccagttgcaa taaaatagta ttgaaagtga
aaaaaaaaaa a
Human NeuroD1 protein sequence (NP_002491)
(SEQ ID NO: 10)
mtksysesgl mgepqpqgpp swtdeclssq deeheadkke ddleamnaee dslrnggeee dededleeee eeeeedddqk
pkrrgpkkkk mtkarlerfk lrrmkanare rnrmhglnaa ldnlrkvvpc ysktqklski etlrlaknyi walseilrsg
kspdlvsfvq tlckglsqpt tnlvagclql nprtflpeqn qdmpphlpta sasfpvhpys yqspglpspp ygtmdsshvf
hvkppphays aalepffesp ltdctspsfd gplspplsin gnfsfkheps aefeknyaft mhypaatlag aqshgsifsg
taaprceipi dnimsfdshs hhervmsaql naifhd
Mouse NeuroD1 nucleic acid sequence (NM_010894)
(SEQ ID NO: 11)
acgaggaatt cgcccacgca gaaggcaagg tgtcccgagg ctccagggtt atgagatcgt cactattcag aaccttttaa
caacaggaag tggaaacatg accaaatcat acagcgagag cgggctgatg ggcgagcctc agccccaagg tcccccaagc
tggacagatg agtgtctcag ttctcaggac gaggaacacg aggcagacaa gaaagaggac gagcttgaag ccatgaatgc
agaggaggac tctctgagaa acgggggaga ggaggaggag gaagatgagg atctagagga agaggaggaa gaagaagagg
aggaggagga tcaaaagccc aagagacggg gtcccaaaaa gaaaaagatg accaaggcgc gcctagaacg ttttaaatta
aggcgcatga aggccaacgc ccgcgagcgg aaccgcatgc acgggctgaa cgcggcgctg gacaacctgc gcaaggtggt
accttgctac tccaagaccc agaaactgtc taaaatagag acactgcgct tggccaagaa ctacatctgg gctctgtcag
agatcctgcg ctcaggcaaa agccctgatc tggtctcctt cgtacagacg ctctgcaaag gtttgtccca gcccactacc
aatttggtcg ccggctgcct gcagctcaac cctcggactt tcttgcctga gcagaacccg gacatgcccc cgcatctgcc
aaccgccagc gcttccttcc cggtgcatcc ctactcctac cagtcccctg gactgcccag cccgccctac ggcaccatgg
acagctccca cgtcttccac gtcaagccgc cgccacacgc ctacagcgca gctctggagc ccttctttga aagcccccta
actgactgca ccagcccttc ctttgacgga cccctcagcc cgccgctcag catcaatggc aacttctctt tcaaacacga
accatccgcc gagtttgaaa aaaattatgc ctttaccatg cactaccctg cagcgacgct ggcagggccc caaagccacg
gatcaatctt ctcttccggt gccgctgccc ctcgctgcga gatccccata gacaacatta tgtctttcga tagccattcg catcatgagc
gagtcatgag tgcccagctt aatgccatct ttcacgatta gaggcacgtc agtttcacta ttcccgggaa acgaatccac
tgtgcgtaca gtgactgtcc tgtttacaga aggcagccct tttgctaaga ttgctgcaaa gtgcaaatac tcaaagcttc aagtgatata
tgtatttatt gtcgttactg cctttggaag aaacagggga tcaaagttcc tgttcacctt atgtattgtt ttctatagct cttctatttt
aaaaataata atacagtaaa gtaaaaaaga aaatgtgtac cacgaatttc gtgtagctgt attcagatcg tattaattat ctgatcggga
taaaaaaaat cacaagcaat aattaggatc tatgcaattt ttaaactagt aatgggccaa ttaaaatata tataaatata tatttttcaa
ccagcatttt actacctgtg acctttccca tgctgaatta ttttgttgtg attttgtaca gaatttttaa tgacttttta taacgtggat
ttcctatttt aaaaccatgc agcttcatca atttttatac atatcagaaa agtagaatta tatctaattt atacaaaata atttaactaa
tttaaaccag cagaaaagtg cttagaaagt tattgcgttg ccttagcact tctttcttct ctaattgtaa aaaagaaaag aaaagaaaaa
aaaccaacaa attgcacaat ttgagcaatt catctcactt taaagttttt cctgctcgct ccctaaaata gaaaccagac ccataacact
caagaggatg aaaaccgaaa tgcattcctt atcaaaacac atcaattcat tacttgcaca agcttgtaaa tacatattat aaataaatgc
caacacacac tcctttaaat caaaagctgc ttgactatca catacaattt gcactctttc tttttagtct tttacttctt tgaattccat
gattttacgg agtgtttgaa gatattgatg tttccagaaa atataaatgc atgattttat acatagtcaa acaaatggtg gtttgtcatc
tattcatgta ataaatttga gcctaaattt attcaggttg ttaatgttgg gtttttatac ctgtgtagtc agttagtaca gtagtttaaa
taaaattcaa accatcgaat tcataattag aacaatagct gttgcatgta aaatgcagtc cagaataagt gctgtttgag atgtgatgct
ggtactactg gaattgacat gtactgtaat cttgtttgta atcctgtgta ttatggtgta atgcacaatt tagaaaactc ccatgcagtt
gcaataaaaa tagtatggaa aatc
Mouse NeuroD1 protein sequence (NP_035024)
(SEQ ID NO: 12)
mtksysesgl mgepqpqgpp swtdeclssq deeheadkke deleamnaee dslrnggeee eededleeee eeeeeeedqk
pkrrgpkkkk mtkarlerfk lrrmkanare rnrmhglnaa ldnlrkvvpc ysktqklski etlrlaknyi walseilrsg
kspdlvsfvq tlckglsqpt tnlvagclql nprtflpeqn pdmpphlpta sasfpvhpys yqspglpspp ygtmdsshvf
hvkppphays aalepffesp ltdctspsfd gplspplsin gnfsfkheps aefeknyaft mhypaatlag pqshgsifss
gaaaprceip idnimsfdsh shhervmsaq lnaifhd
Human CK19 promoter nucleic acid sequence (NM_002276)
(SEQ ID NO: 13)
agatatccgc ccctgacacc attcctccct tcccccctcc accggccgcg ggcataaaag gcgccaggtg agggcctcgc
cgctcctccc gcgaatcgca gcttctgaga ccagggttgc tccgtccgtg ctccgcctcg ccatgacttc ctacagctat
cgccagtcgt cggccacgtc gtccttcgga ggcctgggcg gcggctccgt gcgttttggg ccgggggtcg cctttcgcgc
gcccagcatt cacgggggct ccggcggccg cggcgtatcc gtgtcctccg cccgctttgt gtcctcgtcc tcctcggggg
cctacggcgg cggctacggc ggcgtcctga ccgcgtccga cgggctgctg gcgggcaacg agaagctaac catgcagaac
ctcaacgacc gcctggcctc ctacctggac aaggtgcgcg ccctggaggc ggccaacggc gagctagagg tgaagatccg
cgactggtac cagaagcagg ggcctgggcc ctcccgcgac tacagccact actacacgac catccaggac ctgcgggaca
agattcttgg tgccaccatt gagaactcca ggattgtcct gcagatcgac aatgcccgtc tggctgcaga tgacttccga
accaagtttg agacggaaca ggctctgcgc atgagcgtgg aggccgacat caacggcctg cgcagggtgc tggatgagct
gaccctggcc aggaccgacc tggagatgca gatcgaaggc ctgaaggaag agctggccta cctgaagaag aaccatgagg
aggaaatcag tacgctgagg ggccaagtgg gaggccaggt cagtgtggag gtggattccg ctccgggcac cgatctcgcc
aagatcctga gtgacatgcg aagccaatat gaggtcatgg ccgagcagaa ccggaaggat gctgaagcct ggttcaccag
ccggactgaa gaattgaacc gggaggtcgc tggccacacg gagcagctcc agatgagcag gtccgaggtt actgacctgc
ggcgcaccct tcagggtctt gagattgagc tgcagtcaca gctgagcatg aaagctgcct tggaagacac actggcagaa
acggaggcgc gctttggagc ccagctggcg catatccagg cgctgatcag cggtattgaa gcccagctgg gcgatgtgcg
agctgatagt gagcggcaga atcaggagta ccagcggctc atggacatca agtcgcggct ggagcaggag attgccacct
accgcagcct gctcgaggga caggaagatc actacaacaa tttgtctgcc tccaaggtcc tctgaggcag caggctctgg
ggcttctgct gtcctttgga gggtgtcttc tgggtagagg gatgggaagg aagggaccct tacccccggc tcttctcctg
acctgccaat aaaaatttat ggtccaaggg aaaaaaaaaa aaaaaaaaaa
Human CK19 promoter protein sequence (NP_002267)
(SEQ ID NO: 14)
mtsysyrqss atssfgglgg gsvrfgpgva frapsihggs ggrgvsyssa rfvsssssga ygggyggvlt asdgllagne
kltmqnlndr lasyldkvra leaangelev kirdwyqkqg pgpsrdyshy yttiqdlrdk ilgatiensr ivlqidnarl
aaddfrtkfe teqalrmsve adinglrrvl deltlartdl emqieglkee laylkknhee eistlrgqvg gqvsvevdsa
pgtdlakils dmrsqyevma eqnrkdaeaw ftsrteelnr evaghteqlq msrsevtdlr rtlqgleiel qsqlsmkaal
edtlaetear fgaqlahiqa lisgieaqlg dvradserqn qeyqrlmdik srleqeiaty rsllegqedh ynnlsaskvl

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,323,165: Production of Pancreatic Islet Cells and Delivery of Insulin.
U.S. Pat. No. 7,374,390: GLP-1 Gene Delivery for the Treatment of Type 2 Diabetes.
1. Shapiro A M, Lakey J R, Ryan E A, et al. Islet transplantation in seven patients with type 1 diabetes mellitus using a glucocorticoid-free immunosuppressive regimen. N Engl J Med 343:230, 2000
2. Matsumoto S, Okitsu T, Iwanaga Y, et al. Insulin independence after living-donor distal pancreatectomy and islet allotransplantation. Lancet 365:1642, 2005
3. Bonner-Weir S, Taneja M, Weir G C, et al: In vitro cultivation of human islets from expanded ductal tissue. Proc Natl Acad Sci USA 97:7999, 2000
4. Noguchi H, Bonner-Weir S, Wei F Y, et al: BETA2/NeuroD protein can be transduced into cells due to an arginine- and lysine-rich sequence. Diabetes 54:2859, 2005
5. Noguchi H, Kaneto H, Weir G C, et al: PDX-1 protein containing its own antennapedia-like protein transduction domain can transduce pancreatic duct and islet cells. Diabetes 52:1732, 2003.
6. Yamamoto T, Yamato E, Taniguchi H, et al: Stimulation of cAMP signaling allows isolation of clonal pancreatic precursor cells from adult mouse pancreas. Diabetologia 49:2359, 2006
7. Inada A, Nienaber C, Katsuta H, et al: Carbonic anhydrase II-positive pancreatic cells are progenitors for both endocrine and exocrine pancreas after birth. Proc Natl Acad Sci USA 105:19915, 2008
8. Noguchi H, Xu G, Matsumoto S, Kaneto H, et al. Induction of pancreatic stem/progenitor cells into insulin-producing cells by adenoviral-mediated gene transfer technology. Cell Transplant 15:929, 2006
9. Noguchi H, Matsushita M, Matsumoto S, et al. Mechanism of PDX-1 protein transduction. Biochem Biophys Res Commun 332:68, 2005
10. Hao E, Tyrberg B, Itkin-Ansari P, et al. Beta-cell differentiation from nonendocrine epithelial cells of the adult human pancreas. Nat Med 12:310, 2006
11. Kagaya M, Kaneko S, Ohno H, et al. Cloning and characterization of the 5′-flanking region of human cytokeratin 19 gene in human cholangiocarcinoma cell line. J Hepatol 35:504, 2001
12. Matsumoto S, Noguchi H, Shimoda M, et al. Seven consecutive successful clinical islet isolations with pancreatic ductal injection. Cell Transplant in press
13. Matsumoto S, Okitsu T, Iwanaga Y, et al. Successful islet transplantation from non-heart-beating donor pancreata using modified Ricordi islet isolation method. Transplantation 82:460, 2006
14. Matsumoto S, Noguchi H, Naziruddin B, et al. Improvement of pancreatic islet cell isolation for transplantation. Proc (Bayl Univ Med Cent) 20:357, 2007
15. Noguchi H, Ikemoto T, Naziruddin B, et al. Iodixanol-controlled density gradient during islet purification improves recovery rate in human islet isolation. Transplantation 87:1629, 2009
16. Ikemoto T, Noguchi H, Shimoda M, et al. Islet cell transplantation for the treatment of type 1 diabetes in the USA. J Hepatobiliary Pancreat Surg 16:118, 2009
17. Matsumoto S, Noguchi H, Hatanaka N, et al. Estimation of Donor Usability for Islet Transplantation in the United States with the Kyoto Islet Isolation Method. Cell Transplant 18:549, 2009
18. Naya F J, Stellrecht C M, Tsai M J. Tissue-specific regulation of the insulin gene by a novel basic helix-loop-helix transcription factor. Genes Dev 9:1009, 1995
19. Naya F J, Huang H P, Qiu Y, et al. Diabetes, defective pancreatic morphogenesis, and abnormal enteroendocrine differentiation in BETA2/neuroD-deficient mice. Genes Dev 11:2323, 1997
20. Kojima H, Fujimiya M, Matsumura K, et al. NeuroD-betacellulin gene therapy induces islet neogenesis in the liver and reverses diabetes in mice. Nat Med 9:596, 2003
21. Murdoch T B, McGhee-Wilson D, Shapiro A M, et al. Methods of human islet culture for transplantation. Cell Transplant 13:605, 2004
22. Tyrberg B, Andersson A, Borg L A. Species differences in susceptibility of transplanted and cultured pancreatic islets to the beta-cell toxin alloxan. Gen Comp Endocrinol 122:238, 2001.

What is claimed is:

1. A method of treating diabetes in a mammal in need thereof comprising administering a therapeutically effective amount of an isolated nucleic acid including a sequence encoding a NeuroD1 gene to one or more non-endocrine pancreatic epithelial cells of the mammal and expressing a NeuroD1 protein in the cell, wherein the NeuroD1 protein comprises the amino acid sequence set forth in SEQ ID NOS: 10 or 12, thereby treating the diabetes of the mammal.

2. The method of claim 1, wherein the non-endocrine pancreatic epithelial cells are cytokeratin 19+ positive cells.

3. The method of claim 1, wherein the non-endocrine pancreatic epithelial cells are human cytokeratin 19+ positive cells.

4. The method of claim 1, wherein the NeuroD1 is a human NeuroD1.

5. The method of claim 1, wherein the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a gas and the pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells.

6. The method of claim 5, wherein the gas is a perfluorocarbon gas.

7. The method of claim 5, wherein the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

8. The method of claim 5, wherein the inducible promoter comprises a CK19 promoter.

9. The method of claim 8, wherein the CK19 promoter is a human CK19 promoter.

10. A gene construct comprising:
an isolated nucleic acid including a sequence encoding a NeuroD1 gene, wherein the NeuroD1 gene expresses a NeuroD1 protein comprising the amino acid sequence set forth in SEQ ID NOS: 10 or 12 in one or more cells; and
a constitutive promoter sequence or an inducible promoter sequence operably linked to the NeuroD1 gene.

11. The construct of claim 10, wherein the NeuroD1 is a human NeuroD1.

12. The construct of claim 10, wherein the inducible promoter is a human CK19 promoter.

13. The construct of claim 10, wherein the one or more cells are human cytokeratin 19+ non-endocrine pancreatic epithelial cells.

14. A composition for islet transplantation comprising one or more human cytokeratin 19+ non-endocrine pancreatic epithelial cells, wherein the cells are transfected with a NeuroD1 gene under the control of a constitutive promoter sequence or an inducible promoter sequence operably linked to the NeuroD1 gene.

15. The composition of claim 14, wherein the NeuroD1 gene expresses a NeuroD1 protein comprising the amino acid sequence set forth in SEQ ID NOS: 10 or 12 in the one or more cells.

16. The composition of claim 14, wherein the composition is used for treating diabetes, for promoting euglycemia or for making one or more glucose responsive cells.

17. A composition for making sugar responsive cells comprising a microbubble capable of delivering to non-endocrine pancreatic epithelial cells one or more isolated nucleic acids comprising a plasmid DNA encoding a NeuroD1 gene under the control of a constitutive promoter sequence or an inducible promoter sequence and expressing a NeuroD1 protein in the cells, wherein the NeuroD1 protein comprises the amino acid sequence set forth in SEQ ID NOS: 10 or 12, wherein the microbubbles comprise lipids that release the plasmid by ultrasound disruption into the non-endocrine pancreatic epithelial cells.

18. The composition of claim 17, wherein the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a gas and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells.

19. The composition of claim 18, wherein the gas is a perfluorocarbon gas.

20. The composition of claim 18, wherein the inducible promoter is a CK19 promoter.

21. The composition of claim 20, wherein the CK19 promoter is a human CK19 promoter.

22. The composition of claim 17, wherein the efficacy of NeuroD1 expression is determined by increased responsiveness to blood sugar as measured by insulin release by the non-endocrine pancreatic epithelial cells.

23. The composition of claim 17, wherein the NeuroD1 is a human NeuroD1.

24. A method of treating diabetes or promoting euglycemia in a patient comprising the steps of:
identifying the patient in need of treatment against the diabetes or promotion of the euglycemia; and
injecting an effective amount of a microbubble capable of delivering to non-endocrine pancreatic epithelial cells one or more isolated nucleic acids comprising a plasmid DNA encoding a NeuroD1 gene, wherein the microbubbles comprise lipids that release the plasmid by ultrasound disruption into the non-endocrine pancreatic epithelial cells.

25. The method of claim 24, wherein the non-endocrine pancreatic epithelial cells are cytokeratin 19+ positive cells.

26. The method of claim 24, wherein the non-endocrine pancreatic epithelial cells are human cytokeratin 19+ positive cells.

27. The method of claim 24, wherein the NeuroD1 is a human NeuroD1.

28. The method of claim 24, wherein the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a gas and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells.

29. The method of claim 28, wherein the gas is a perfluorocarbon gas.

30. The method of claim 28, wherein the inducible promoter comprises a CK19 promoter.

31. The method of claim 30, wherein the CK19 promoter is a human CK19 promoter.

32. The method of claim 28, wherein the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

33. A method of providing an insulin-producing cell, the method comprising:
providing one or more isolated non-endocrine pancreatic epithelial cell;
transfecting the cells with an isolated nucleic acid encoding a NeuroD1 polypeptide comprising a sequence that is at least 95% identical to SEQ ID NOS: 10 or 12, wherein the polypeptide can increase, cause transcription or both of the insulin gene in the non-endocrine pancreatic epithelial cell; and
assaying insulin production in the cells, thereby providing an insulin-producing cell.

34. The method of claim 33, wherein the non-endocrine pancreatic epithelial cells are cytokeratin 19+ positive cells.

35. The method of claim 33, wherein the non-endocrine pancreatic epithelial cells are human cytokeratin 19+ positive cells.

36. The method of claim 33, wherein the isolated nucleic acid is delivered via ultrasound-targeted microbubble destruction (UTMD) using a vector comprising one or more pre-assembled liposome naked plasmid DNA (pDNA) microbubble complexes, wherein the microbubble comprises a lipid shell enclosing a gas and a pDNA comprising a constitutive promoter sequence or an inducible promoter sequence operably linked to the NeuroD1 gene, wherein an ultrasound disruption of the one or more microbubbles in the pancreas delivers the NeuroD1 gene into the non-endocrine pancreatic epithelial cells.

37. The method of claim 36, wherein the gas is a perfluorocarbon gas.

38. The method of claim 36, wherein the inducible promoter comprises the CK19 promoter.

39. The method of claim 38, wherein the CK19 promoter is a human CK19 promoter.

40. The method of claim 36, wherein the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

41. The method of claim 33, wherein the NeuroD1 is a human NeuroD1.

42. An insulin producing cell generated by the method of claim 33

43. A method of treating one or more non-endocrine pancreatic epithelial cells, islets, or both transplanted in a liver with an ultrasound-targeted microbubble destruction (UTMD) technique, wherein the treatments results in increased insulin production, increased glucose responsiveness or both comprising the step of delivering via ultrasound-targeted microbubble destruction (UTMD) a vector comprising one or more pre-assembled liposome naked plasmid DNA (pDNA) microbubble complexes, wherein the microbubble comprises a lipid shell enclosing a gas and the pDNA comprising a constitutive promoter sequence or an inducible promoter sequence operably linked to a NeuroD1 gene, wherein an ultrasound disruption of the one or more microbubbles in the pancreas delivers the NeuroD1 gene into the transplanted non-endocrine pancreatic epithelial cells, islets or both resulting in an expression of a NeuroD1 protein in the non-endocrine pancreatic epithelial cells, islets or both, wherein the NeuroD1 protein comprises the amino acid sequence set forth in SEQ ID NOS: 10 or 12.

44. The method of claim 43, further comprising the step of determining an increased responsiveness to blood sugar by measuring an insulin release by the transplanted non-endocrine pancreatic epithelial cells, the islets or both.

45. The method of claim 43, wherein the efficacy of the transplantation of the one or more non-endocrine pancreatic epithelial cells, islets, or both is measured by improved revascularization, improved islet cell function, increased vessel density or combinations thereof.

46. The method of claim 43, wherein the NeuroD1 is a human NeuroD1.

47. The method of claim 43, wherein the inducible promoter is a human CK19 promoter.

48. The method of claim 43, wherein the gas is a perfluorocarbon gas.

49. The method of claim 43, wherein the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

50. A non-endocrine pancreatic epithelial cell, an islet or both with increased glucose responsiveness, increased insulin production or both made by the method of claim 43.


SEQUENCE LISTING (14 sequences)

SEQ ID NO: 1 - DNA, 20 bp, Artificial Sequence
  Synthetic oligonucleotide.
ctccatcctggcctcgctgt

SEQ ID NO: 2 - DNA, 20 bp, Artificial Sequence
  Synthetic oligonucleotide.
gctgtcaccttcaccgttcc

SEQ ID NO: 3 - DNA, 20 bp, Artificial Sequence
  Synthetic oligonucleotide.
cgagcagaaccggaaggatg

SEQ ID NO: 4 - DNA, 21 bp, Artificial Sequence
  Synthetic oligonucleotide.
agccgctggtactcctgattc

SEQ ID NO: 5 - DNA, 18 bp, Artificial Sequence
  Synthetic oligonucleotide.
gcgctcaggcaaaagccc

SEQ ID NO: 6 - DNA, 20 bp, Artificial Sequence
  Synthetic oligonucleotide.
gccattgatgctgagcggcg

SEQ ID NO: 7 - DNA, 20 bp, Artificial Sequence
  Synthetic oligonucleotide.
cagccgcagcctttgtgaac

SEQ ID NO: 8 - DNA, 20 bp, Artificial Sequence
  Synthetic oligonucleotide.
aatgccacgcttctgcaggg

SEQ ID NO: 9 - DNA, 2641 bp, Homo sapiens
gagaacggggagcgcacagcctggacgcgtgcgcaggcgtcaggcgcatagacctgctagcccctcagctagcggccccgcccgcgcttagcatcactaactgggctatataacctgagcgcccgcgcggccacgacacgaggaattcgcccacgcaggaggcgcggcgtccggaggccccagggttatgagactatcactgctcaggacctactaacaacaaaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaggcacgccagtttcaccatttccgggaaacgaacccactgtgcttacagtgactgtcgtgtttacaaaaggcagccctttgggtactactgctgcaaagtgcaaatactccaagcttcaagtgatatatgtatttattgtcattactgcctttggaagaaacaggggatcaaagttcctgttcaccttatgtattattttctatagctcttctatttaaaaaataaaaaaatacagtaaagtttaaaaaatacaccacgaatttggtgtggctgtattcagatcgtattaattatctgatcgggataacaaaatcacaagcaataattaggatctatgcaatttttaaactagtaatgggccaattaaaatatatataaatatatatttttcaaccagcattttactacttgttacctttcccatgctgaattattttgttgtgattttgtacagaatttttaatgactttttataatgtggatttcctattttaaaaccatgcagcttcatcaatttttatacatatcagaaaagtagaattatatctaatttatacaaaataatttaactaatttaaaccagcagaaaagtgcttagaaagttattgtgttgccttagcacttctttcctctccaattgtaaaaaaaaaaaaaaaaaaaaaaaaaaaaaaaaaattgcacaatttgagcaattcatttcactttaaagtctttccgtctccctaaaataaaaaccagaatcataattttcaagagaagaaaaaattaagagatacattccctatcaaaacatatcaattcaacacattacttgcacaagcttgtatatacatattataaataaatgccaacatacccttctttaaatcaaaagctgcttgactatcacatacaatttgcactgttactttttagtcttttactcctttgcattccatgattttacagagaatctgaagctattgatgtttccagaaaatataaatgcatgattttatacatagtcacaaaaatggtggtttgtcatatattcatgtaataaatctgagcctaaatctaatcaggttgttaatgttgggatttatatctatagtagtcaattagtacagtagcttaaataaattcaaaccatttaattcataattagaacaatagctattgcatgtaaaatgcagtccagaataagtgctgtttgagatgtgatgctggtaccactggaatcgatctgtactgtaattttgtttgtaatcctgtatattatggtgtaatgcacaatttagaaaacattcatccagttgcaataaaatagtattgaaagtgaaaaaaaaaaaa

SEQ ID NO: 10 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 11 - DNA, 2494 bp, Mus musculus
acgaggaattcgcccacgcagaaggcaaggtgtcccgaggctccagggttatgagatcgtcactattcagaaccttttaacaacaggaagtggaaacatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattagaggcacgtcagtttcactattcccgggaaacgaatccactgtgcgtacagtgactgtcctgtttacagaaggcagcccttttgctaagattgctgcaaagtgcaaatactcaaagcttcaagtgatatatgtatttattgtcgttactgcctttggaagaaacaggggatcaaagttcctgttcaccttatgtattgttttctatagctcttctattttaaaaataataatacagtaaagtaaaaaagaaaatgtgtaccacgaatttcgtgtagctgtattcagatcgtattaattatctgatcgggataaaaaaaatcacaagcaataattaggatctatgcaatttttaaactagtaatgggccaattaaaatatatataaatatatatttttcaaccagcattttactacctgtgacctttcccatgctgaattattttgttgtgattttgtacagaatttttaatgactttttataacgtggatttcctattttaaaaccatgcagcttcatcaatttttatacatatcagaaaagtagaattatatctaatttatacaaaataatttaactaatttaaaccagcagaaaagtgcttagaaagttattgcgttgccttagcacttctttcttctctaattgtaaaaaagaaaagaaaagaaaaaaaaccaacaaattgcacaatttgagcaattcatctcactttaaagtttttcctgctcgctccctaaaatagaaaccagacccataacactcaagaggatgaaaaccgaaatgcattccttatcaaaacacatcaattcattacttgcacaagcttgtaaatacatattataaataaatgccaacacacactcctttaaatcaaaagctgcttgactatcacatacaatttgcactctttctttttagtcttttacttctttgaattccatgattttacggagtgtttgaagatattgatgtttccagaaaatataaatgcatgattttatacatagtcaaacaaatggtggtttgtcatctattcatgtaataaatttgagcctaaatttattcaggttgttaatgttgggtttttatacctgtgtagtcagttagtacagtagtttaaataaaattcaaaccatcgaattcataattagaacaatagctgttgcatgtaaaatgcagtccagaataagtgctgtttgagatgtgatgctggtactactggaattgacatgtactgtaatcttgtttgtaatcctgtgtattatggtgtaatgcacaatttagaaaactcccatgcagttgcaataaaaatagtatggaaaatc

SEQ ID NO: 12 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 13 - DNA, 1490 bp, Homo sapiens
agatatccgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaa

SEQ ID NO: 14 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL